(19) United States
(12) Patent Application Publication
(10) Pub. No.: US 2004/0136493 A1
(43) Pub. Date: Jul. 15, 2004

(54) Xray detector having tiled photosensitive modules and Xray system

(76) Inventors: Yasutaka Konno, Kokubunji (JP); Kenichi Okajima, Mitaka (JP); Hironori Ueki, Hachioji (JP)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/616,356

(22) Filed: Jul. 10, 2003

(30) Foreign Application Priority Data
Jan. 15, 2003 (JP) 2003-007275

(51) Int. Cl. G21K 1/12; H05G 1/60; A61B 6/00; G01N 23/00

(52) U.S. Cl. 378/19

(57) ABSTRACT

There are provided an X-ray detector which can realize a larger area without lowering resolution and reducing X-ray detective efficiency when obtaining a matrix construction having a large number of X-ray detecting elements by tiling and a system using the same.

An X-ray detector 104 has a construction in which a plurality of photo-electric modules 111 having a plurality of X-ray detecting elements 110 located in a two-dimensional manner are pasted onto a distribution module 113. The X-ray detecting element 110 has scintillators 112, transparent means 121 and photo-electric means 114. These are optically connected to each other. On the edge of the transparent means 121 on one of the photo-electric modules 111 mounted on the distribution module to be adjacent to each other is formed a cutaway part 120 so that the area of an output surface 211 outputting a light to the photo-electric means 114 is smaller than that of an incident surface 210 upon which a light is incident from the scintillators 112. A space caused by the cutaway part 120 is located wiring between the photo-electric module 111 and the distribution module 113 or wiring between the photo-electric modules 111 adjacent to each other.

### BACKGROUND OF THE INVENTION AND RELATED ART STATEMENT FIELD OF THE INVENTION

The present invention relates to an X-ray detector and an X-ray system using the same. More specifically, the present invention relates to an X-ray detector of a two-dimensional (2D) array type which has X-ray detecting elements located in a matrix which irradiate an X-ray onto a scintillator to change it to an optical signal and further convert the optical signal to an electric signal for detection and an X-ray system which uses the same in an X-ray CT apparatus and an X-ray imaging system.

The case of applying an X-ray detector to an X-ray CT apparatus will be described below as a representative example. The X-ray CT apparatus is an apparatus which can obtain a cross-sectional view of an object and is widely used in fields of medical and non-destructive inspection. The configuration of an X-ray CT apparatus as an example used in medical is shown in the schematic diagram of FIG. 2 and will be described according to this.

As shown in FIG. 2, the X-ray CT apparatus has an X-ray source 100, X-ray detectors 104, a data acquisition system (DAS) 118, a central processor 105, an image display unit 106, input means 119, a controller 117, a rotated gantry 101, and a bed 103. A plurality of the X-ray detectors 104-k are located in an arc substantially centered at the X-ray source 100 and are mounted on the rotated gantry 101 together with the X-ray source 100. Here, k is the number of the X-ray detectors 104 and k is 1, 2, . . . , as shown in FIG. 2. Using this, the X-ray detector 104 having number k is expressed as 104-k. For simplifying the description, FIG. 2 shows the case that k is 8 at the maximum. In an actual apparatus, generally, k is, e.g., about 40 at the maximum.

Imaging and processing methods of the X-ray CT apparatus will be described. When there is a start input from the input means 119, an X-ray in a fan is irradiated from the X-ray source 100 onto the object 102 placed on the bed 103. The X-ray transmitted through the object 102 is converted to an electric signal (projection) by the X-ray detectors 104.

The imaging is repeated by rotating the rotated gantry 101 in a rotating direction 108 to change the irradiation angle of the X-ray to the object 102. Projections for 360° are acquired. The projections are imaged, e.g., every 0.4°. The controller 117 controls rotation of the rotated gantry 101 and readout of the X-ray detectors 104.

The projections thus obtained are acquired by the data acquisition system 118. To the projections are added convolution processing and back projection processing by the central processor 105. The tomographic images of distribution of X-ray attenuation coefficient of the object 102 are reconstructed. This result is displayed on the image display unit 106.

The schematic diagram of the X-ray detector 104-k is shown in FIG. 3. A direction 108 (hereinafter, referred to as a channel direction) of FIG. 3 coincides with the rotating direction 108 of FIG. 2. A direction 107 (hereinafter, referred to as a slice direction) coincides with the direction of axis of rotation 107 of FIG. 2.

As shown in FIG. 3, the X-ray detector 104 has a construction in which a plurality of X-ray detecting elements 110 converting an X-ray to an electric signal are provided on a distribution module 113 in a matrix. i (=1, 2) is the number of the slice direction (indicating a column in the direction of axis of rotation) of the X-ray detecting element 110. j (=1, 2) is the number of the channel direction (indicating a line in the rotating direction) thereof. The X-ray detecting element 110 is expressed as 110-i-j.

Other elements, e.g., scintillators 112 and photo-electric means 114 are expressed in the same manner. The X-ray detecting element 110-i-j has the scintillator 112-i-j absorbing an X-ray to convert it to a light and the photo-electric means 114-i-j changing the light to an electric signal. These are optically connected to each other. The photo-electric means 114-i-j is formed on a photo-electric module (semi-conductor module) 111. For simplifying the description, in FIG. 3, i, j of the X-ray detecting elements 110 are expressed as 2 at the maximum. In general, the X-ray detecting elements 110 in 24 columns for i and 2 lines for j are located.

The X-ray CT apparatus is broadly divided by the number of lines of the direction of axis of rotation (slice direction) 107 of the detectors. The X-ray CT apparatus having one line of the detectors is called a single-slice type. The X-ray CT apparatus having a plurality of lines of the detectors is called a multi-slice type. When the above imaging is performed by the X-ray CT apparatus of a single-slice type, only one tomographic image can be obtained in a slice surface vertical to the axis of rotation. When obtaining tomographic images in a large number of slice surfaces, the slice surfaces are changed in the direction of axis of rotation 107 to perform the same imaging in the respective moving positions.

To substantially realize such imaging, the prior art X-ray CT apparatus is rotatably driven, and in parallel, continuously moves the bed 103 in the direction of axis of rotation 107. This is called spiral scanning. In this method, projections can be acquired in a large number of slice surfaces and 3D tomographic images can be reconstructed.

The X-ray CT apparatus of a multi-slice type can image projections in a large number of slice surfaces when not performing spiral scanning. When performing imaging while spiral scanning is conducted in the direction of axis of rotation 107 at the same sampling intervals, the imaging can be performed in a short time as compared with the X-ray CT apparatus of a single-slice type. When the same imaging range is imaged in the same imaging time, imaging can be performed at small sampling intervals as compared with the X-ray CT apparatus of a single-slice type.

As described above, the multi-slice type has a great advantage. The X-ray CT apparatus of a multi-slice type is widely used. In recent years, a multi-slice X-ray CT apparatus in which the number of lines of the X-ray detectors is 4 or above has appeared. The number of lines of the X-ray detectors tends to be increased. The following Patent Document 1 is given as the X-ray CT apparatus of a multi-slice type.

[Patent Document 1]

Japanese Laid-Open No. 2001-24225

### OBJECTS AND SUMMARY OF THE INVENTION

To manufacture a plurality of lines of X-ray detectors to be mounted on the X-ray CT apparatus of a multi-slice type, a large semi-conductor wafer is necessary. When the semi-conductor wafer is larger, the price itself of the wafer is high. The number of detectors which can be manufactured from one semi-conductor wafer is reduced. A high technique for handling is required. The yield of manufacturing the detectors is decreased. The cost for manufacturing the X-ray detectors is high.

As a method for solving the problems, there is a method in which a plurality of X-ray detector modules provided with a small number of lines of the X-ray detecting elements 110 are close to each other for use. Such method can manufacture substantially multi-slice X-ray detector modules at relatively low cost.

In this method, when the X-ray detector module is surrounded by other modules, a method for reading out a signal of the module is a problem. A dead space is caused between the photo-electric modules 111 adjacent to each other for wiring from the photo-electric module 111 to the distribution module 113. The resolution is lowered in the position.

As a method for solving such problem, Patent Document 1 (Japanese Laid-Open No. 2001-242253) proposes an X-ray detector which permits readout when an X-ray detector module is surrounded by other modules by performing wiring between a distribution module and an X-ray detector module for reading out a signal from the X-ray detector module using a space provided by cutting away part of a scintillator. The scintillator is provided with a cutaway part, lowering the X-ray detective efficiency in the photo-electric means.

In separating processing for corresponding the scintillator 112 with the photo-electric means (photo-electric element) 114 formed on the photo-electric module 111, a diamond cutter or a multiwire saw is used. A high technique is necessary for processing the cutaway part of the micro scintillator.

To solve the above prior art problems, an object of the present invention is to provide an X-ray detector which can easily perform wiring connection between a photo-electric module and a distribution module or wiring connection between photo-electric modules adjacent to each other without making the cutaway part of the micro scintillator for wiring connection so as to realize a matrix construction having a large area without lowering resolution, causing a dead space and reducing X-ray detective efficiency.

To achieve the above object, in an X-ray detector of the present invention, when an X-ray irradiated onto a scintillator is converted into a light produced by the scintillator and the light is converted to an electric signal by phot-electric means, a light output surface of the scintillator is not directly connected to photo-electric means of a photo-electric module and is connected to the photo-electric module via transparent means having a specific construction transmitting the light.

The feature of the X-ray detector according to the present invention is in the construction of transparent means provided between a scintillator and photo-electric means. In a square part including of the transparent means at least a light output surface of the transparent means which is positioned on the edge of a photo-electric module is provided a cutaway part. The area of the light output surface emitted to the side of the photo-electric means is smaller than that of a light input surface upon which a light is incident from the scintillator. The light incident from the scintillator is focused without waste to be incident upon the photo-electric means.

In the square part including the light output surface of the transparent means is provided the cutaway part. A space provided on the edge of the photo-electric module is a space for wiring.

In the space are provided wiring between a distribution module for reading out a signal from an X-ray detector module and the photo-electric module of the X-ray detector or/and wiring between the photo-electric modules adjacent to each other (The wirings are called module wiring means.). It is possible to realize an X-ray detector having a matrix construction having a large area without lowering resolution, causing a dead space and reducing X-ray detective efficiency when tiling a plurality of the X-ray detector modules without cutting away the scintillator unlike the prior art.

### BRIEF DESCRIPTION OF THE DRAWINGS

FIG. 1 is a perspective view showing the construction of an X-ray detector 104 of the present invention described in Example 1;

FIG. 2 is a system configuration diagram of an X-ray CT apparatus of a prior art;

FIG. 3 is a perspective view showing the construction of an X-ray detector 104 of the prior art;

FIG. 4 is a circuit diagram of the X-ray detector 104 of the present invention described in Example 1;

FIG. 5 is a 3D circuit diagram of the X-ray detector 104 of the present invention described in Example 1;

FIG. 6 is a top view of the X-ray detector 104 shown in FIG. 1;

FIG. 7 is a cross-sectional view taken along line A-A′ of the X-ray detector 104 shown in FIG. 6;

FIG. 8A is a perspective view showing manufacturing process 1-1 of the X-ray detector 104 of the present invention described in Example 1;

FIG. 8B is a perspective view showing manufacturing process 1-2 of the X-ray detector 104 of the present invention described in Example 1;

FIG. 9A is a perspective view showing manufacturing process 1-3 of the X-ray detector 104 of the present invention described in Example 1;

FIG. 9B is a perspective view showing manufacturing process 1-4 of the X-ray detector 104 of the present invention described in Example 1;

FIG. 10A is a perspective view showing manufacturing process 2-1 of the X-ray detector 104 of the present invention described in Example 1;

FIG. 10B is a perspective view showing manufacturing process 2-2 of the X-ray detector 104 of the present invention described in Example 1;

FIG. 10C is a perspective view showing manufacturing process 2-3 of the X-ray detector 104 of the present invention described in Example 1;

FIG. 11A is a perspective view showing manufacturing process 2-4 of the X-ray detector 104 of the present invention described in Example 1;

FIG. 11B is a perspective view showing manufacturing process 2-5 of the X-ray detector 104 of the present invention described in Example 1;

FIG. 11C is a perspective view showing manufacturing process 2-6 of the X-ray detector 104 of the present invention described in Example 1;

FIG. 12A is a perspective view showing manufacturing processes 2-7 to 2-8 of the X-ray detector 104 of the present invention described in Example 1;

FIG. 12B is a perspective view showing manufacturing process 2-8 of the X-ray detector 104 of the present invention described in Example 1;

FIG. 13 is a cross-sectional view showing a modification of the shape of a cutaway part 120 shown in FIG. 7;

FIG. 14 is a cross-sectional view showing a modification of the shape of the cutaway part 120 shown in FIG. 7;

FIG. 15 is a cross-sectional view showing a modification of the shape of the cutaway part 120 shown in FIG. 7;

FIG. 16 is a circuit diagram of the X-ray detector 104 of the present invention described in Example 2;

FIG. 17 is a 3D circuit diagram of the X-ray detector 104 of the present invention described in Example 2;

FIG. 18 is a cross-sectional view taken along line B-B′ of the X-ray detector 104 shown in FIG. 16;

FIG. 19A is a perspective view showing manufacturing process 1-1 of the X-ray detector 104 of the present invention described in Example 2;

FIG. 19B is a perspective view showing manufacturing process 1-2 of the X-ray detector 104 of the present invention described in Example 2;

FIG. 19C is a perspective view showing manufacturing process 1-3 of the X-ray detector 104 of the present invention described in Example 2;

FIG. 20 is a system configuration diagram of an X-ray CT apparatus of the present invention described in Example 3; and

FIG. 21 is a system configuration diagram of an X-ray CT apparatus of the present invention described in Example 4.

### DETAILED DESCRIPTION OF PREFERRED EMBODIMENT

Embodiments specifically showing the features of the present invention will be described below.

1. An X-ray detector according to the present invention has:

(1) an X-ray sensitive module having a plurality of X-ray detecting elements having a scintillator converting an X-ray to a light and transparent means optically connected to a light output surface of the scintillator transmitting an output light from the scintillator located integrally in a two-dimensional manner via optical reflecting means in a first and a second directions;

(2) a photo-electric module in which photo-electric means located in a two-dimensional manner corresponding to the transparent means of the X-ray detecting elements converting an output light outputted from the scintillator via the transparent means to an electric signal, a first data line reading out the electric signal, a first addressing line addressing the photo-electric means reading out the electric signal, and electrode pads forming part of the first data line or/and the first addressing line are formed, a light output surface of the transparent means is optically connected to the photo-electric means, the area of the photo-electric means positioned on the edge in the first direction is formed to be smaller than that of the photo-electric means positioned in other positions, the electrode pads are formed near an end surface on which the transparent means is not mounted, and a plurality of the X-ray sensitive modules are mounted to be adjacent to each other in the first or the second direction;

(3) a distribution module in which a second data line connected to the first data line reading out the electric signal and a second addressing line connected to the first addressing line addressing the photo-electric means reading out the electric signal are formed, and a plurality of the photo-electric modules are mounted; and

(4) module wiring means electrically connecting the electrode pads of the photo-electric modules adjacent to each other, or/and the electrode pad and the second data line, or/and the electrode pad and the second addressing line.

In the construction, the X-ray detector of the present invention can realize a matrix construction having a large area having a large number of X-ray detecting elements without lowering resolution, causing a dead space and reducing X ray detective efficiency.

The cutaway part provided in the transparent means allows a plurality of the photo-electric modules to be tiled on the distribution module so that the photo-electric means are at equal intervals without providing a dead space. The thickness of the scintillator is uniform regardless of the X-ray detecting elements. The X-ray detective efficiency is not reduced.

In the X-ray detector according to the present invention, preferably, a plurality of the photo-electric modules are pasted onto the surface of the distribution module in the second direction. The electrode pads as part of the readout means or/and the control means are provided on the surface of the photo-electric module on the edge in the second direction. Such construction can realize a matrix construction having a large area, not only in the first direction, but also in the second direction.

2. In the X-ray detector according to the present invention, preferably, the transparent means is made of a resin layer which has a thickness smaller than that of the scintillator, has optical transmittance higher than that of the scintillator and is stable to an X-ray, and has a shape in which an angle θ of a normal vector at an arbitrary point of a surface except for a light input surface from the scintillator and an output surface of the resin layer and a normal vector of the input surface or the output surface is 45°≦θ<90°. In such construction, a light incident from the scintillator is reflected on the side surface of the transparent means and is hard to return to the incident surface. The transparent means can efficiently transmit the light.

3. An X-ray detector according to the present invention preferably has:

(1) an X-ray sensitive module having a plurality of X-ray detecting elements having a scintillator converting an X-ray to a light and transparent means optically connected to an output surface of the scintillator transmitting an output light from the scintillator located integrally in a two-dimensional manner via optical reflecting means in a first and a second directions, the transparent means positioned on the edge in said first direction having a cutaway part in part thereof;

(2) a photo-electric module in which photo-electric means located in a two-dimensional manner corresponding to the transparent means of the X-ray detecting elements converting an output light outputted from the scintillator via the transparent means to an electric signal, a first data line reading out the electric signal, a first addressing line addressing the photo-electric means reading out the electric signal, and electrode pads forming part of the first data line or/and the first addressing line are formed, a light output surface of the transparent means is optically connected to the photo-electric means, the area of the photo-electric means positioned on the edge in the first direction is formed to be smaller than that of the photo-electric means positioned in other positions, the electrode pads are formed near an end surface on which the transparent means is not mounted, and a plurality of the X-ray sensitive modules are mounted to be adjacent to each other in the first or the second direction;

(3) a distribution module in which a second data line connected to the first data line reading out the electric signal and a second addressing line connected to the first addressing line addressing the photo-electric means reading out the electric signal are formed, and a plurality of the photo-electric modules are-mounted; and

(4) module wiring means electrically connecting the electrode pads of the photo-electric modules adjacent to each other, or/and the electrode pad and the second data line, or/and the electrode pad and the second addressing line.

4. An X-ray CT apparatus according to the present invention has: an X-ray tube generating an X-ray; a plurality of X-ray detectors according to any one of claims 1 to 3 located in an arc in the second direction opposite the first X-ray tube; a detector control circuit producing a control signal for addressing the photo-electric means reading out the electric signal of the X-ray detector and inputting it to the second addressing line; a data acquisition system acquiring the electric signals outputted from the second data line to convert them to digital data; arithmetic processing means performing arithmetic processing the digital data; and an image display unit displaying the result of the arithmetic processing. In such construction, the X-ray CT apparatus on which the X-ray detectors are mounted to obtain a tomographic image of an object can be realized.

5. In the X-ray CT apparatus, preferably, the data acquisition system has data correcting means correcting the analog electric signal from the photo-electric means corresponding to part or all of the X-ray detecting elements, or the digital data obtained by converting the analog electric signal. In such construction, the X-ray CT apparatus on which the X-ray detectors are mounted to obtain a tomographic image of an object can be realized.

6. An X-ray imaging system according to the present invention has: an X-ray tube generating an X-ray; one or more X-ray detectors according to any one of claims 1 to 3 located opposite the X-ray tube; a detector control circuit producing a control signal for addressing the photo-electric means reading out the electric signal of the X-ray detector and inputting it to the second addressing line; a data acquisition system acquiring the electric signals outputted from the second data line to convert them to digital data; and an image display unit displaying the digital data. In such construction, the X-ray imaging system on which the X-ray detectors are mounted to obtain a projection of an object can be realized.

In the X-ray imaging system, the data acquisition system has data correcting means correcting the analog electric signal from the photo-electric means corresponding to part or all of the X-ray detecting elements of the X-ray detector, or the digital data obtained by converting the analog electric signal. In such construction, the X-ray imaging system on which the X-ray detectors are mounted to obtain a projection of an object can be realized.

In the X-ray CT apparatus or the X-ray imaging system according to the present invention, preferably, the data acquisition system has data correcting means correcting data of at least part of the X-ray detecting elements. This can perform correcting processing of correction of variation in the characteristic of the X-ray detecting elements and the data acquisition system, correction of variation in distribution of irradiated X-ray, noise reduction of an image filter, and interpolation processing of output values.

### EXAMPLE

Examples of the present invention will be specifically described below according to the drawings.

### Example 1

(1) Construction of an X-Ray Detector 104:

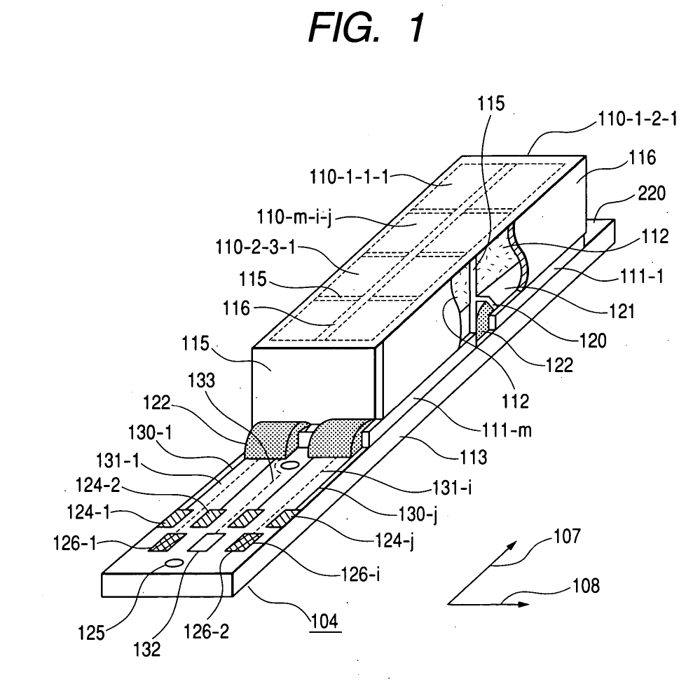

A construction example of the X-ray detector 104 as Example 1 of the present invention will be described using FIG. 1 and FIGS. 4 to 7.

FIG. 1 shows the construction of the X-ray detector 104 according to the present invention. In this example, for simplifying the description, for convenience, the X-ray detector 104 having two photo-electric modules 111 mounted on a distribution module 113 is shown. For simplifying the description, the X-ray detector 104 also has X-ray detecting elements 110 arrayed in four lines and two columns. The array of the number j of lines and the number i of columns of the X-ray detecting elements 110 constructing the X-ray detector 104 in the present invention is not limited to this example.

The X-ray detector 104 of FIG. 1 has a circuit module 113, photo-electric modules 111, transparent means 121 and scintillators 112. m (=1, 2) expresses an array number of the photo-electric modules 111. The photo-electric module 111 having number m is expressed as 111-m. i and j express a matrix. i (=1, 2) is an array number of the X-ray detecting elements in a channel direction. j (=1, 2) expresses an array number thereof in a slice direction. The transparent means 121 positioned in the number i (indicating the column) in the channel direction and the number j (indicating the line) in the slice direction in the X-ray detector 104 and is on the photo-electric module 111-m is expressed as 121-m-i-j. The scintillators 112 and the X-ray detecting elements 110 are expressed in the same manner.

A plurality of photo-electric means (photodiodes) 114-m-i-j are formed on the photo-electric module 111-m in a matrix corresponding to the number of the X-ray detecting elements. The two photo-electric modules 111 are pasted onto a top surface 220 of the distribution module 113. The number of the photo-electric modules 111 are for simplifying the description and does not limit the present invention.

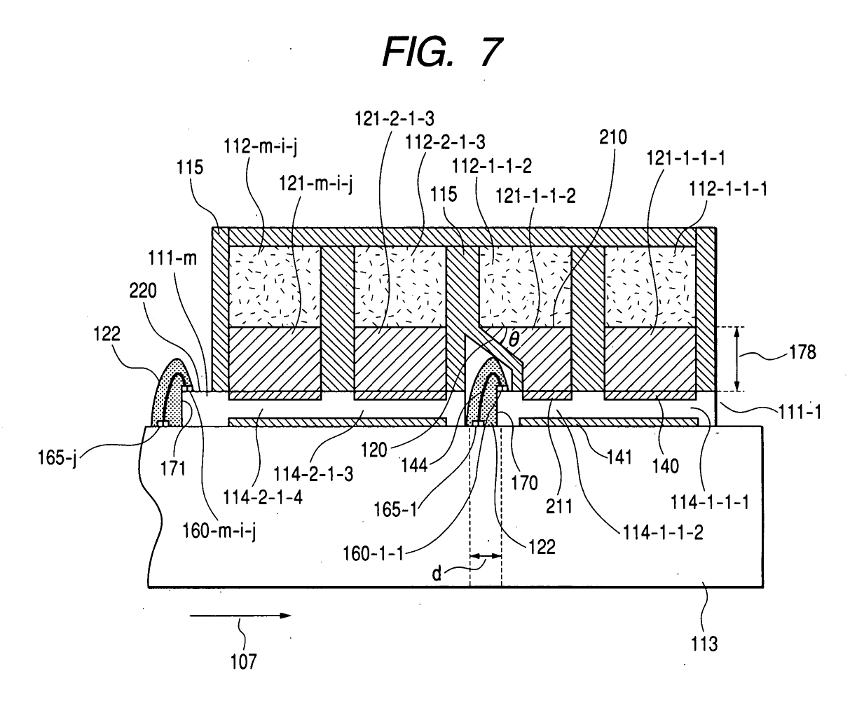

The photo-electric means 114-m-i-j, the transparent means 121-m-i-j and the scintillator 112-m-i-j construct the X-ray detecting element 110-m-i-j (for more detail, see the cross-sectional view of FIG. 7). When an X-ray is incident upon the X-ray detecting element 110-m-i-j, the X-ray is converted to a light by the scintillator 112-m-i-j. The light is incident upon the transparent means 121-m-i-j optically connected to the scintillator 112-m-i-j.

The transparent means 121-m-i-j is made of an optical transmitting material which is transparent to the light produced by the scintillator 112-m-i-j, has an optical transmittance higher than that of the scintillator 112-m-i-j, and is stable to an X-ray (No coloring and crack is caused.). In this example, a mold of an epoxy resin is used. The thickness is e.g., about 500 to 2000 μm and is preferably smaller than that of the scintillator 112.

The scintillators 112 and the transparent means 121-m-i-j are separated by separators 116 in a channel direction 108. The separators 116 prevent cross-talk between the scintillators 112 adjacent to each other and between the transparent means 121-m-i-j and have on its surface optical reflectivity to enhance the focusing efficiency.

The scintillators 112 and the transparent means 121-m-i-j are separated by optical separators 115 in a slice direction 107 and the incident surface. The optical separators also prevent cross-talk to enhance focusing efficiency. A light is incident upon the photo-electric means 114-m-i-j optically connected to the transparent means 121-m-i-j to produce an electric signal by the photo-electric means 114-m-i-j.

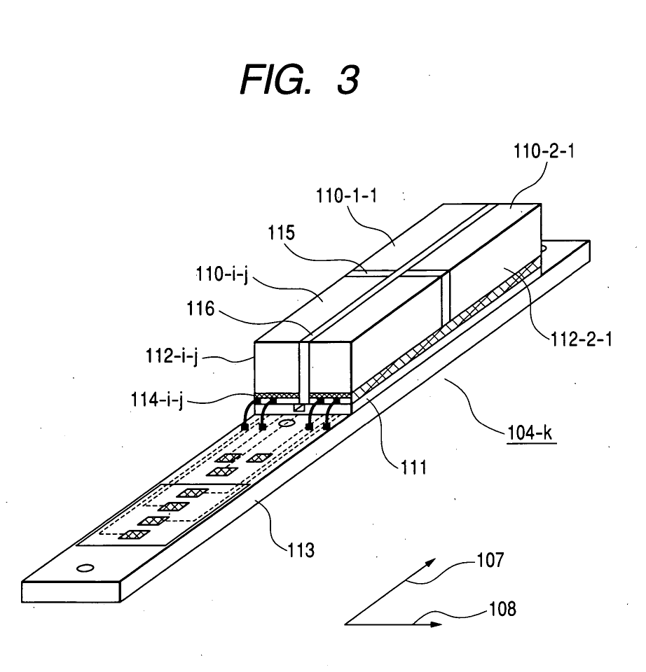

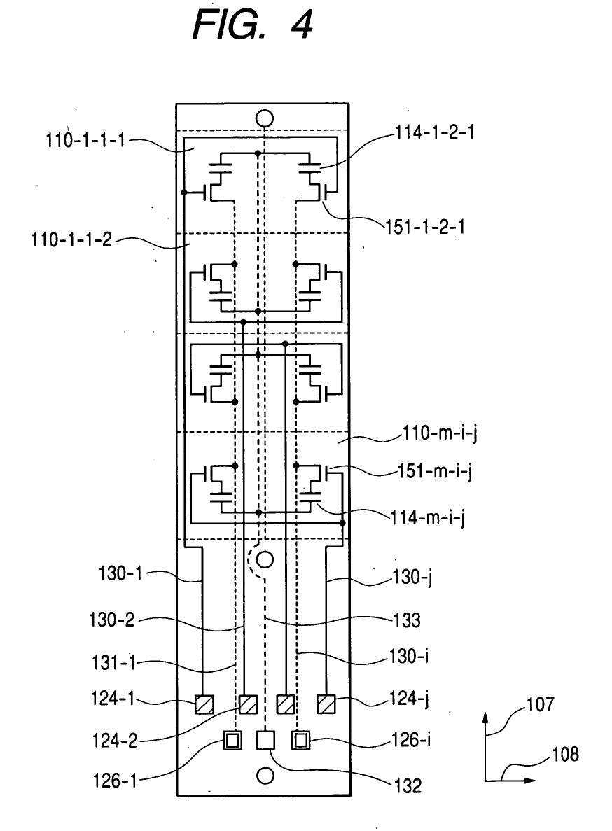

Signal readout will be described. A circuit diagram when the X-ray detector 104 shown in FIG. 1 is seen from the top is shown in FIG. 4. The electric signal produced as described above is stacked into the photo-electric means 114-m-i-j.

One of electrodes of the photo-electric means 114-m-i-j is electrically connected to an electrode pad for ground line 132 by a ground line 133. The other electrode is connected to the drain electrode of a switching element 151-m-i-j formed on the photo-electric module 111-m for each of the X-ray detecting elements 110-m-i-j.

The source electrode of the switching element 151-m-i-j is electrically connected to a pad for data line 126-i by a data line 131-i for each of the photo-electric means 114-m-i-j positioned in the common column i. The gate electrode of the switching element 151-m-i-j is electrically connected to a pad for address line 124-j by an addressing line 130-j for each of the photo-electric means 114-m-i-j positioned in the common line j.

In such construction, when a control signal is inputted to the pad for address line 124-j, a signal of the X-ray detecting element 110-m-i-j positioned in the same line j can be outputted from the pad for data line 126 in parallel.

The pad for address line 124-j inputting the control signal is sequentially switched. The electric signal of the X-ray detecting element 110-m-i-j belonging to the same column i can be sequentially read out from the pad for data line 126-i.

FIG. 5 shows the circuit construction of FIG. 4 in a 3D manner and is a 3D circuit diagram by separating the two photo-electric modules 111-1 and 111-m and the distribution module 113.

As shown in FIG. 5, on the photo-electric module 111-m, a pair of the photo-electric means 114-m-i-j and the switching element 151-m-i-j are located in a matrix.

The gate electrode of the switching element 151-m-i-j belonging to the same line j is electrically connected to an electrode pad for address line 161-m-j on the photo-electric module 111-m for module wiring means. The source electrode of the switching element 151-m-i-j belonging to the same column i is electrically connected to an electrode pad for data Line 160-m-i on the photo-electric module 111-m for module wiring means.

On the distribution module 113, the pad for address-line 124-j is electrically connected to an electrode pad for address line 165-j on the distribution module 113 for module wiring means by the addressing line 130-j. On the same, the pad for data line 126-i is electrically connected to an electrode pad for data line 166-i on the distribution module 113 for module wiring means by the data line 131-i. On the same, the electrode pad for ground line 132 is electrically connected to an electrode pad for ground line 167 on the distribution module 113 by the ground line 133.

When integrating the photo-electric module 111 with the distribution module 113, the electrode pad for data line 166-i on the distribution module 113 is electrically connected to the electrode pad for ground line 167 on the distribution module 113. The electrode pad for address line 161-m-j on the photo-electric module 111 for module wiring means is electrically connected to the electrode pad for address line 165-j on the distribution module 113 for module wiring means by module wiring means. The electrode pad for data line 160-m-i on the photo-electric module 111 for module wiring means is electrically connected to the electrode pad for data line 166-i on the distribution module 113 for module wiring means by module wiring means. The module wiring means is wire bonding.

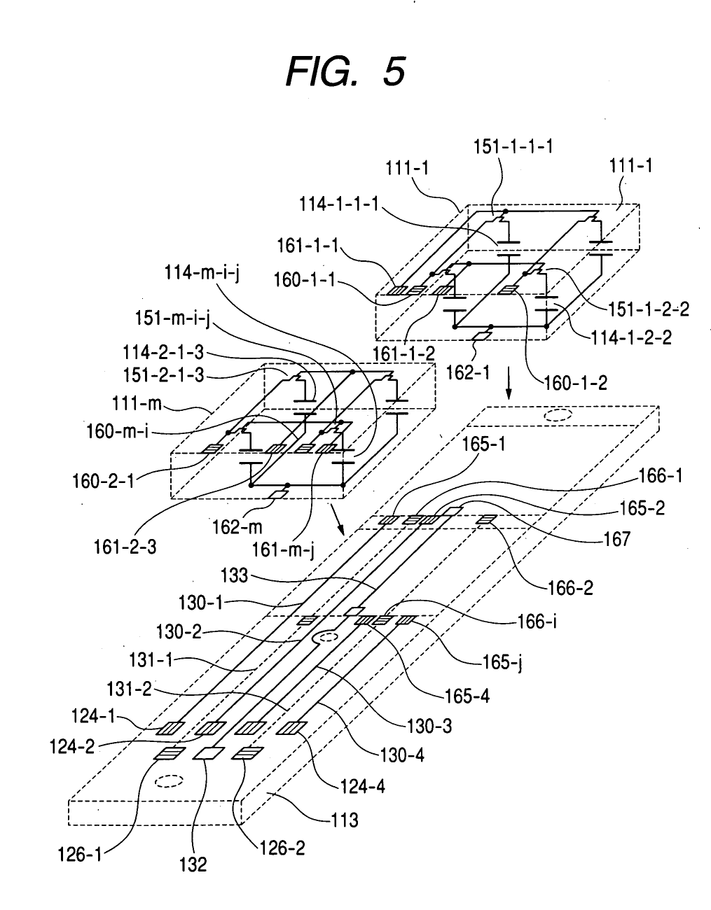

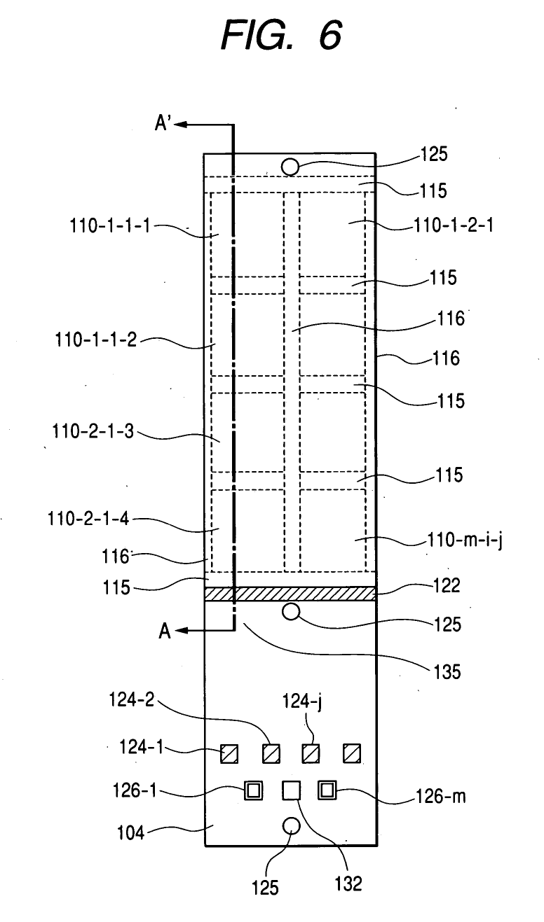

FIG. 6 shows a top view of the X-ray detector 104 according to the present invention. A cross-sectional view taken along line A-A′ of FIG. 6 is shown in FIG. 7. The X-ray detecting element 110-m-i-j shown in FIG. 7 belongs to the same column i. A wiring 144 (hereinafter, called module wiring means) between the modules 113-114 is formed by an edge 170 of the photo-electric module 111-1 and an edge 171 of the adjacent photo-electric module 111-2. The electrode pad for address line 161-m-j on the photo-electric module 111-1 for module wiring means is electrically connected to the electrode pad for address line 165-j on the distribution module 113 for module wiring means. In the drawing, the electrode pad 165-1 on the circuit module 113 is connected to the electrode pad 160-1-1 on the photo-electric module 111-1 by the module wiring means 144.

A space around the periphery of the edge 170 of the photo-electric module 111-1 which can be provided with the module wiring means 144 is realized in such a manner that in the transparent means 121-1-1-2, the area of an output surface 211 of a light to the photo-electric means 114-1-1-2 is smaller than that of an input surface 210 upon which a light is incident from the scintillator 112-1-1-2 and that the area of the photo-electric means 114-1-1-2 is smaller than that of the other photo-electric means 114.

In the construction of the transparent means 121-1-1-2, part of the other transparent means 121 is cut away. The part corresponding to the cutaway part is called a cutaway part 120.

In the construction of the photo-electric means 114, on the top surface 220 and the edge 170 of the photo-electric module 111 are located the electrode pad for address line 161-1-j (j=1, 2) on the photo-electric module 111 for module wiring means and the electrode pad for data line 160-1-i (i=1, 2) on the photo-electric module 111 for module wiring means. The X-ray detecting elements 110 can be located at equal intervals in the slice direction 107.

The construction of the cutaway part 120 provided with a space for forming the module wiring means 144 by cutting away part of the transparent means 121 will be described here. As shown in FIG. 7, the cross-sectional construction of the cutaway part 120 of the transparent means 121 has a linear slope having a tilt angle θ from the substantially top angle of the transparent means 121 to its horizontal top surface (the incident surface 210) and a vertical surface cut down from the lower end part of the slope to the bottom surface substantially vertically. The cutaway position of the bottom surface of the transparent means 121 cut down substantially vertically is the substantially center part of the bottom surface. About half of the bottom surface is cut away from the edge.

The tilt angle θ of the slope of the cutaway part 120 and the incident surface 210 is 45°≦θ<90°. Desirably, the angle θ is as close to 45° as possible to reduce the thickness t of the transparent means 121. In consideration of the thickness t of the transparent means 121, a preferable angle θ is. 45°≦θ≦60°. When the angle θ is less than 45° and an incident light is reflected on the slope of the cutaway part 120, the rate of the light returning to the incident surface is increased so that a light output to the photo-electric element is reduced corresponding to it.

The thickness t of the transparent means 121 is e.g., about 500 to 2000 μm and is desirably below the thickness of the scintillator 112. The distance d between the photo-electric modules 111 adjacent to each other is e.g., 100 to 500 μm.

The module wiring means 144 electrically connects the electrode pad for data line 160-m-i on the photo-electric module 111-m for module wiring means and the electrode pad for data line 165-i on the distribution module 113 for module wiring means.

The X-ray detecting element 110-1-1-2 on the edge is made into such construction. The photo-electric modules 111 can be pasted onto the distribution module 113 without causing a dead space. The multi-slice X-ray detector 104 can be realized without lowering the resolution.

To secure a space for forming the module wiring means 144, the scintillator is cut away in the prior art. In the present invention, no cutaway part is provided in the scintillator and the cutaway part 120 is provided in the transparent means 121. The X-ray attenuation coefficient of the X-ray detecting element is not lower than that of other the X-ray detecting elements. In the prior art, to compensate for the reduced X-ray attenuation coefficient by cutting away the scintillator, an electric compensation circuit is required. In the present invention, since the characteristic of the X-ray detecting elements is uniform, an electric compensation circuit is not required.

The transparent means 121 according to the present invention is preferably made of an epoxy resin. The epoxy resin is hard to lower the optical transmittance by an X-ray. The change with time in sensitivity of the photo-electric means with use can be reduced. The cutaway part 120 is made in the transparent means 121 made of an epoxy resin, which can be performed by a resin forming technique. It is processed more easily than the case of making the cutaway part 120 of the scintillator.

(2) Method for Manufacturing the X-Ray Detector 104:

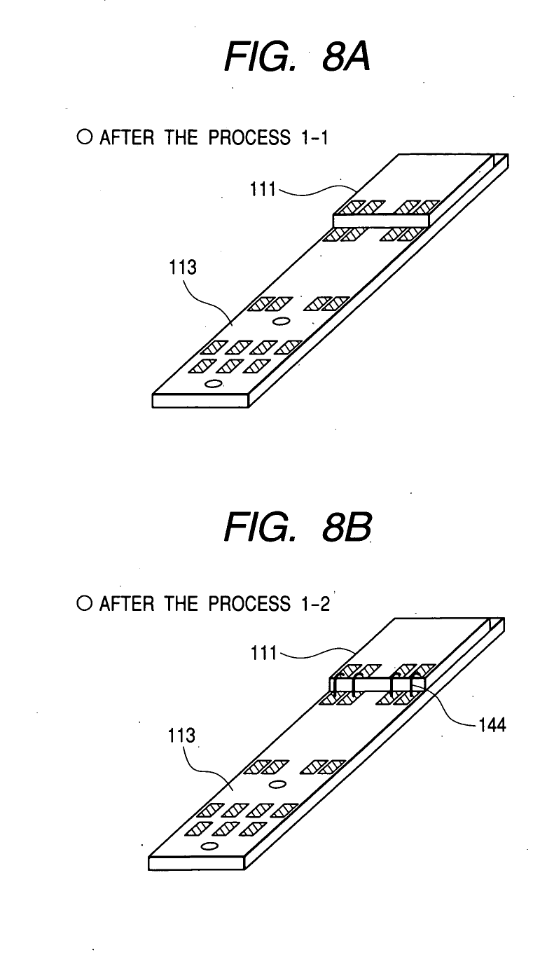

A method for manufacturing the X-ray detector 104 according to the present invention will be described according to FIGS. 8 to 15. The method for manufacturing the X-ray detector 104 shown in this example is an example of the manufacturing method for realizing the X-ray detector 104 of the present invention. This does not limit the method for realizing the present invention.

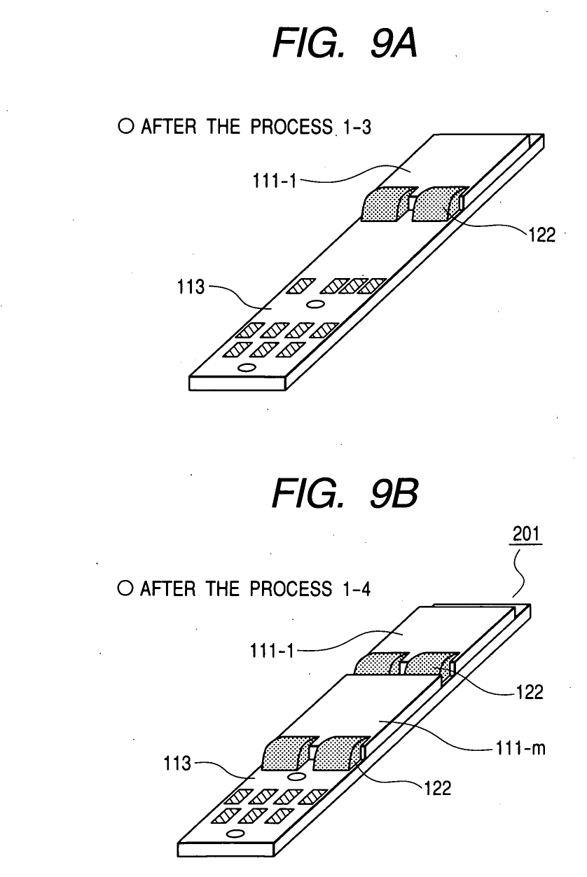

The photo-electric module 111 is pasted onto the distribution module 113 to perform electric connection. This process is shown in the process 1-1 of FIG. 8A to the process 1-4 of FIG. 9B.

In the process 1-1 as shown in FIG. 8A, the photo-electric module 111 is pasted onto the distribution module 113. In this pasting, the electrode pad for ground line 162-m on the photo-electric module 111 is electrically connected to the electrode pad for data line 167 on the distribution module 113 for module wiring means by solder (see FIG. 5). The electrode pad for data line 160 and the electrode pad for address line 161 are provided on the front surface of the photo-electric module 111. The electrode pad for ground line 162 is provided on the back surface thereof.

In the process 1-2 as shown in FIG. 8B, the electrode pad for data line 160-m-i on the photo-electric module 111 for module wiring means is electrically connected to the electrode pad for data line 166-i on the distribution module 113 for module wiring means by the module wiring means 144. The electrode pad for address line 161-m-j on the photo-electric module 111 for module wiring means is electrically connected to the electrode pad for address line 165-j on the distribution module 113 for module wiring means by the module wiring means 144 (see FIG. 5).

In the process 1-3 as shown in FIG. 9A, wiring protective layers 122 are provided to protect the module wiring means 144. The wiring protective layers 122 are insulation.

In the process 1-4 as shown in FIG. 9B, the processes 1-1 to 1-3 are performed to all the photo-electric modules 111. All the photo-electric modules 111 are pasted onto the distribution module 113 for electric connection. A module 201 having the photo-electric modules 111 mounted on the distribution module 113 is a module converting a light to an electric signal.

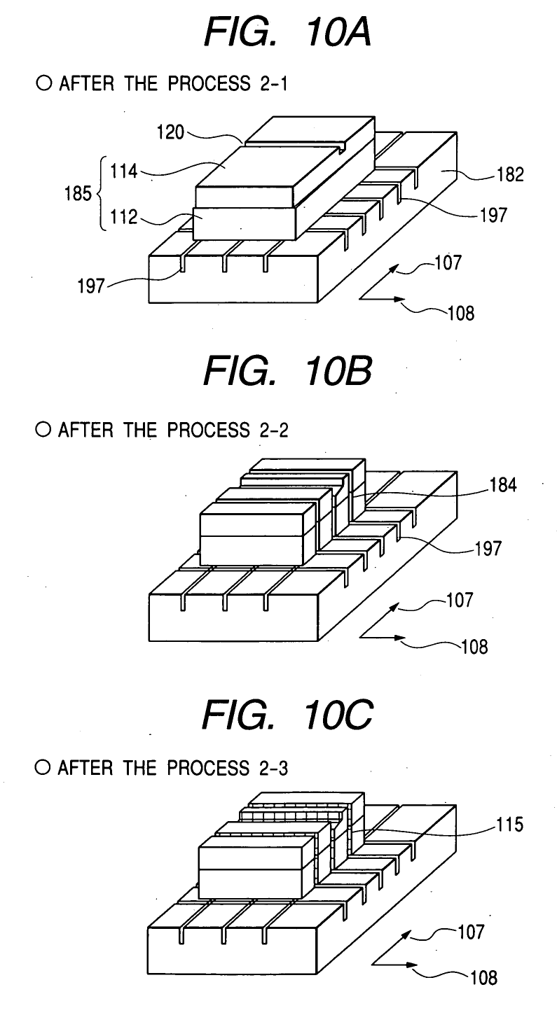

As shown in FIGS. 10A to 12B, an integrated block 185 of the scintillator 112 and the transparent means 114 is manufactured to be pasted onto the photo-electric module 111.

In the process 2-1 as shown in FIG. 10A, the scintillator 112 is fixed onto a support base 182. In the fitting, a bonding agent is used. There is used a bonding agent on a surface 202 of the scintillator 112 which can be easily removed when the scintillator 112 is delaminated from the support base 182 later.

Channels 197 are provided on the support base 182 in the channel direction 108 and the slice direction 107. The size of one lattice realized by the channels 197 in the two directions corresponds to the X-ray detecting element 110. The number of lattices in the two directions corresponds to the number of the X-ray detecting elements 110 of the X-ray detector 104 in the channel direction 108 and the slice direction 107.

The transparent means 114 is bonded onto the scintillator 112. The transparent means 114 is made by hardening an epoxy resin by a hardening agent. It is hardened after placing the epoxy resin into a mold realizing the shape of the cutaway part 120 to form the cutaway part 120.

The pasting position of the transparent means 114 in the slice direction 107 is decided using the positions of the cutaway part 120 and the channel 197. The transparent means 114 is located so that the edge of the cutaway part 120 is positioned on the channel 197. As the bonding agent of the transparent means 114 and the scintillator 112, a bonding agent which optically transparent to a light incident from the scintillator 112 is used.

After the bonding agents are hardened, in the process 2-2 shown in FIG. 10B, the scintillator 112 and the transparent means 114 are separated for each of the channels 197 arrayed in the slice direction 107. The cutting-away is performed by a diamond cutter or a multiwire saw.

In the process 2-3 shown in FIG. 10C, the optical separators 115 are manufactured in channels 184 made in the process 2-2. As the optical reflector used for the optical separators 115, an optical reflector including barium sulfide (BaS) or titanium dioxide (TiO₂) is used. As the optical separator 115, a putty-like optical reflector is used to harden it.

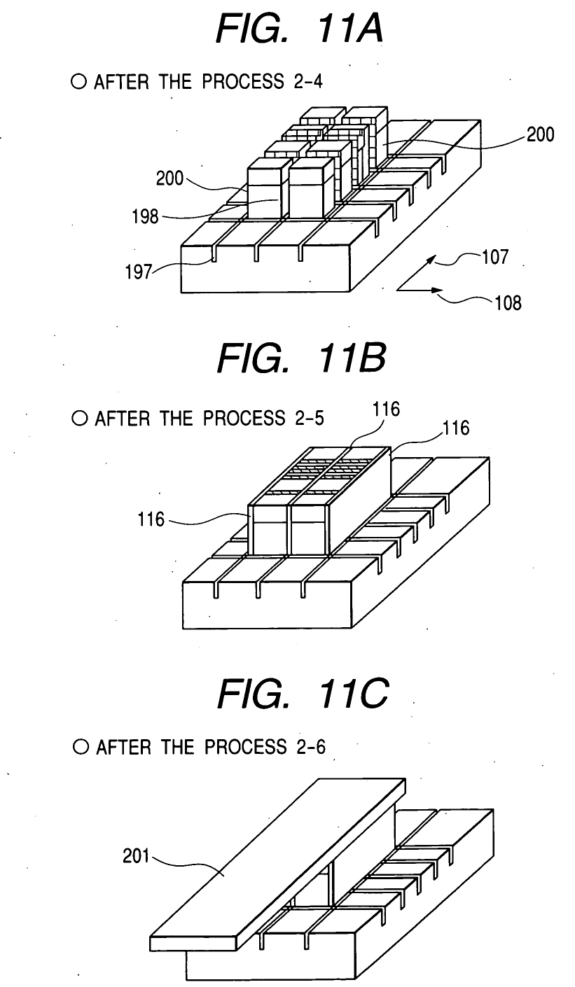

In the process 2-4 shown in FIG. 11A, the scintillator 112 and the transparent means 114 are separated for each of the channels 197 lined in the channel direction 108.

In the process 2-5 shown in FIG. 11B, the separators 116 are provided in the channels 197 made in the process 2-4 and side surfaces 200 on the edge in the channel direction 108 of the X-ray detector 104. The separators 116 are made by molybdenum (Mo), tantalum (Ta), tungsten (W) and lead (Pb) having optical reflectivity, an alloy having these elements as main constituents, or a metal having a large X-ray attenuation coefficient in which the optical separator 115 is coated onto the surface. The thickness is e.g., 100 to 200 μm.

The separators 116 have the same height as that of the scintillator 112 or are projected therefrom. In this example, they are projected from the height of the scintillator 112 and have almost the same height as that of the transparent means 114. The separators 116 are bonded onto the scintillator 112 and the transparent means 114. For the bonding, a bonding agent which is optically transparent to a light incident from the scintillator 112 is used.

In the process 2-6 shown in FIG. 1C, there are pasted the transparent means 114 surface of the block 185 of the scintillator 112 and the transparent means 114 made in the processes 2-1 to 2-5 and the photo-electric module 111 surface of the module 201 integrating the distribution module 113 with the photo-electric module 111 made in the process 1-1 of FIG. 8A to the process 1-4 of FIG. 9B. A marker for positioning for mounting the block 185 is provided in the module 201. In the pasting, the positions of the marker and the channels 197 of the support base 182 are used to perform relative positioning.

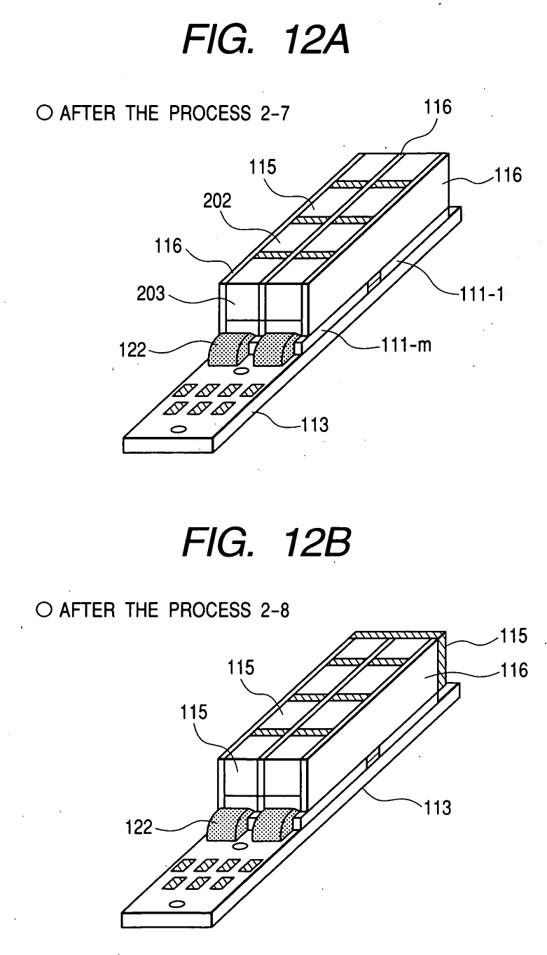

In the process 2-7 shown in FIG. 12A, the support base 182 is delaminated from the scintillator 112 to remove the bonding agent, from the surface 202 of the scintillator 112.

In the process 2-8 shown in FIG. 12B, the optical separators 115 are provided on the top surface and side surface 203 of the scintillator 112. The optical separators 115 are made by coating a liquid optical reflector in which titanium oxide powder is suspended by hardening the same. In such process, the X-ray detector 104 of the present invention shown in FIG. 1 is completed.

In this example, as shown in FIG. 10, the cutaway part 120 provided in the transparent means 114 is realized using the mold (the resin forming technique). The present invention is not limited to this. It can be realized by directly processing the transparent means 114 by lathe.

In this example, the cutaway part 120 is provided only in the transparent means 121 positioned on the edge of the photo-electric module 111 in the slice direction 107. The present invention is not limited to this. It may be provided in the transparent means 121 positioned on the edge of the photo-electric module 111 in the channel direction 108. It may be provided in the transparent means 121 positioned on the edge of the photo-electric module 111 in both the slice direction 107 and the channel direction 108.

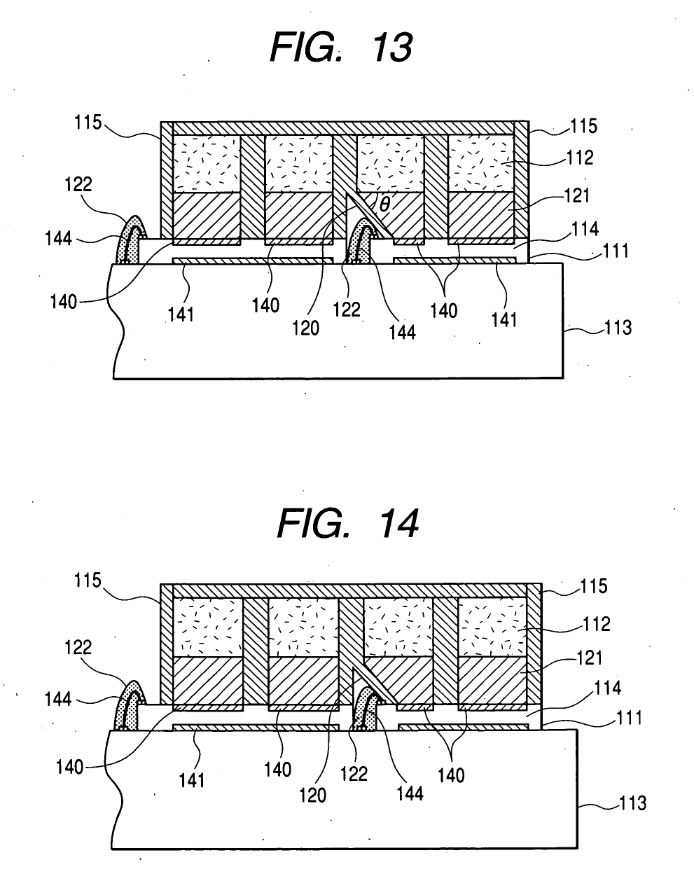

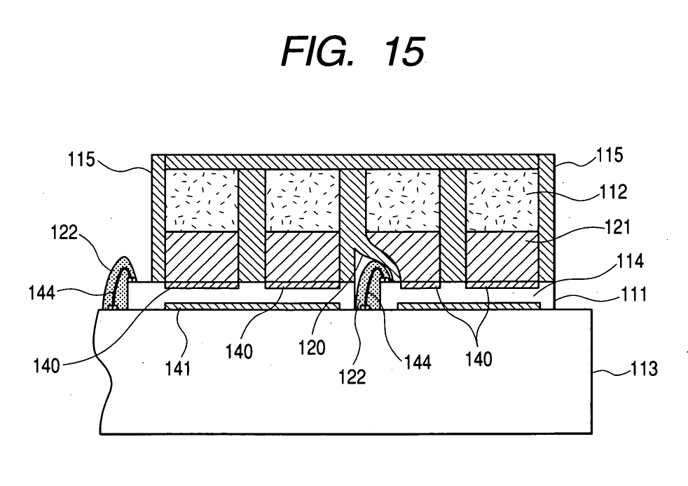

The shape of the cutaway part 120 of the present invention is not limited to the shape of this example. It may be of shape of the cutaway part 120 sloped from the surface contacted to the scintillator 112 onto the photo-electric module 111 as shown in FIG. 13, the cutaway part 120 sloped from the midpoint of the side surface part of the transparent means 121 onto the photo-electric module 111, as shown in FIG. 14, or the cutaway part 120 in which the square of the transparent means 121 is cut away in a curve, as shown in FIG. 15. It is important that in the cutaway part 120, a light outputted from the scintillator 112 is effectively incident upon the photo-electric means without being returned to the scintillator. The light reflected on the cutaway part 120 may be incident onto the photo-electric means without being returned to the scintillator.

In this example, as a method for reducing cross-talk between the scintillators 112 adjacent to each other and the transparent means 121 adjacent to each other, the optical separator 115 is used in the slice direction 108 and the separator 116 is used in the channel direction 107. The present invention is not limited to this. The separator 116 is used in the slice direction 108 and the optical separator 115 is used in the channel direction 107. The optical separator 115 may be used in both the directions. The separator 116 may be used in both the directions. The method may be different by the scintillator 112 and the transparent means 121.

### Example 2

(1) Construction of an X-Ray Detector 104:

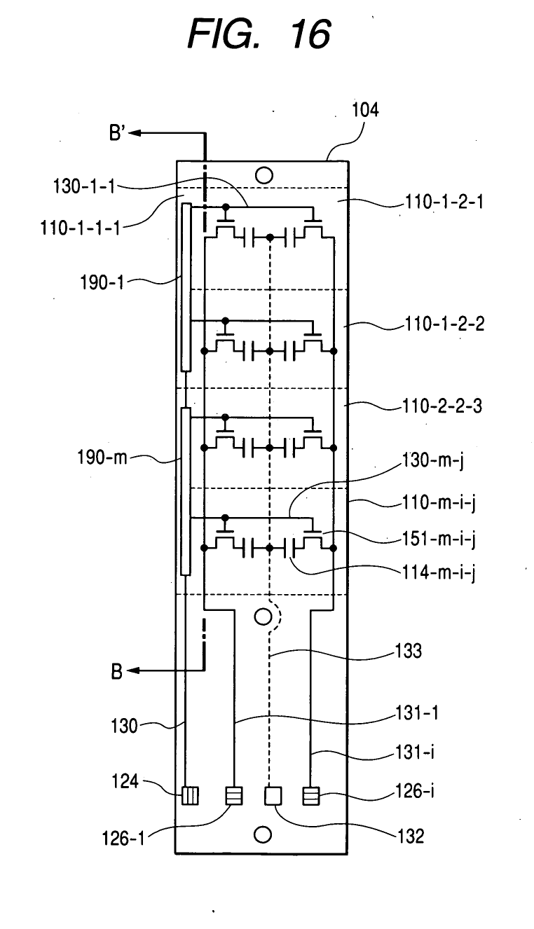

FIG. 16 shows a circuit diagram of the X-ray detector 104 according to the present invention seen from the top. The X-ray detector 104 of this drawing shows the case of X-ray detecting elements 110 in four lines and two columns for simplifying the description.

The X-ray detecting element 110-m-i-j of the X-ray detector 104 has unillustrated scintillators 112-m-i-j, unillustrated transparent means 121-m-i-j, photo-electric means 114-m-i-j and switching elements 151-m-i-j, which are located in a matrix.

The source electrode of the switching element 151-m-i-j of the X-ray detecting element 110-m-i-j belonging to the same column i is electrically connected to an electrode pad for data line 126-i by a common data line 131-i. The gate electrode of the switching electrode 151-m-i-j of the X-ray detecting element 110-m-i-j belonging to the same line j is electrically connected to a vertical shift-resistor 190-m by an addressing line 130-m-j.

The vertical shift-resistor 190-m is electrically connected to an electrode pad for address line 124 by the addressing line 130. A signal starting readout is inputted to the electrode pad for address line 124, the vertical shift-resistor 190-m outputs the signal to the addressing line 130-m-j in the line j to turn on the switching element 151-m-i-j.

The vertical shift-resistor 190-m sequentially switches the line j of the addressing line 130-m-j outputting the signal. From the controls, the X-ray detector 104 realizes parallel readout of the X-ray detecting element 110-m-i-j belonging to the same line j and sequential readout of the X-ray detecting element 110-m-i-j belonging to the same column i.

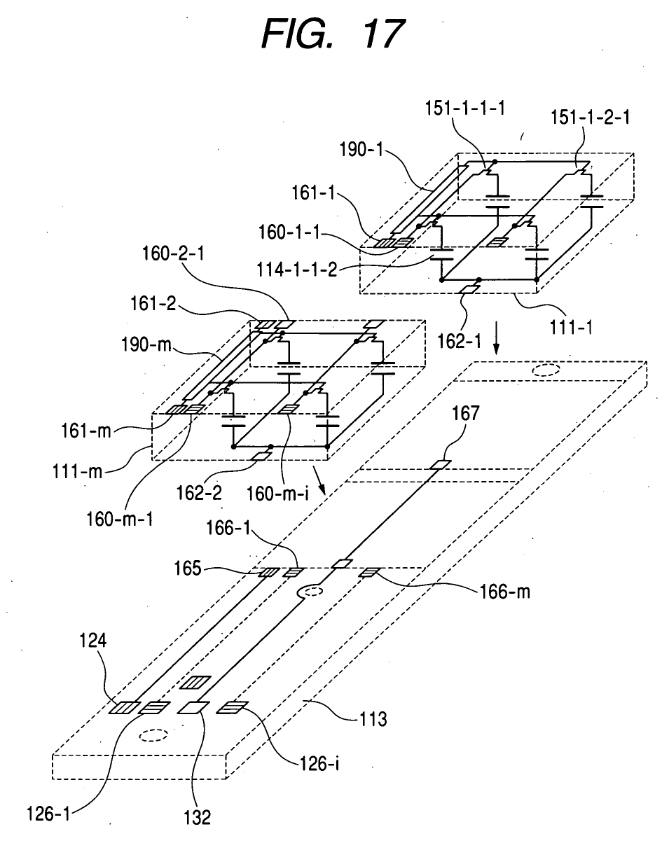

FIG. 17 shows a 3D circuit diagram of a distribution module 113 and photo-electric modules 111 realizing the circuit diagram of FIG. 16. For simplifying the description, the relation between the two photo-electric modules 111-m and 111-1 and the distribution module 113 is shown. In FIG. 17, the numerals of the photo-electric means 114-m-i-j and the switching element 151-m-i-j are omitted. See FIG. 16 for these.

The photo-electric module 111-m has a pair of the photo-electric means 114-m-i-j and the switching element 151-m-i-j, the vertical shift-resistor 190-m controlling the readout, an electrode pad for address line 161-m for inputting/outputting a control signal to/from the vertical shift-resistor 190-m, and an electrode pad for data line 160-m-i for outputting a signal produced by an X-ray.

The distribution module 113 has the electrode pad for address line 124, the electrode pad for data line 126, an electrode pad for ground line 132, an electrode pad for data line 166 on the distribution module 113 for module wiring means, an electrode pad for address line 165 on the distribution module 113 for module wiring means, and an electrode pad for ground line 167 on the distribution module 113.

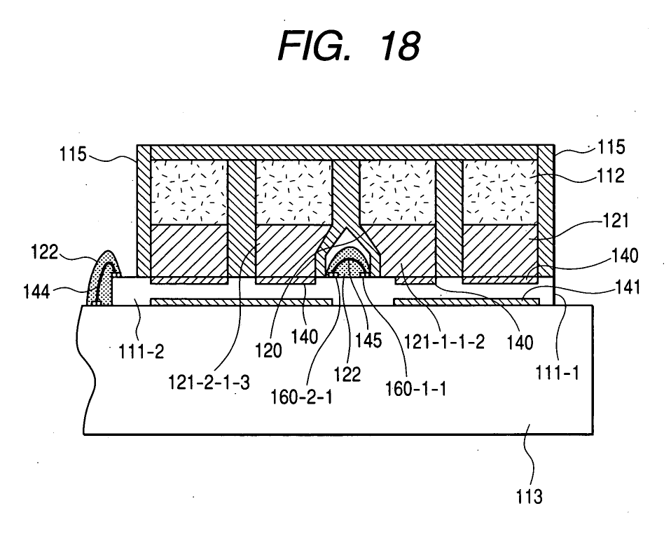

FIG. 18 shows a cross-sectional view taken along line B-B′ of FIG. 16. The electrode pads for data line 160 of the photo-electric modules 111-1 and 111-2 adjacent to each other are connected by wiring 145. The electrode pad for data line 160-1-1 of the photo-electric module 111-1 and the electrode pad for data line 160-2-1 of the photo-electric module 111-2 are electrically connected by the wiring 145.

A space for connecting the electrode pads of the photo-electric modules 111 adjacent to each other by the wiring 145 is a space provided by cutting away the square of the transparent means 121 positioned on the edge region including the electrode pads of the photo-electric modules 111-1 and 111-2 to provide the cutaway part 120.

There is described here that the electrode pads for data line of the photo-electric modules adjacent to each other are connected by the wiring 145. A wiring between the electrode pads for address line is provided in the same manner. The electrode pads for address line 161 for module wiring means of the photo-electric modules 111 adjacent to each other are electrically connected by the wiring 145.

(2) Method for Manufacturing the X-Ray Detector 104:

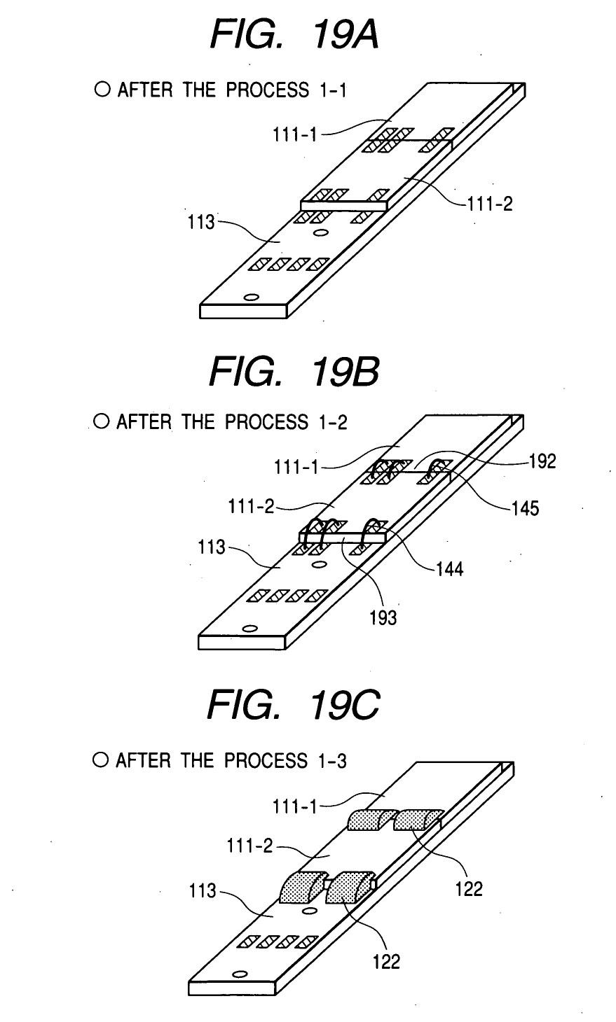

A method for manufacturing the X-ray detector 104 of this example will be described according to a process diagram shown in FIG. 19. The manufacturing method shown here is an example thereof. The process in which the photo-electric module 111-m is pasted onto the distribution module 113 for electric connection is different from the processes 1-1 of FIGS. 8 to 1-4 of FIG. 9 shown in Example 1.

In the process 1-1 shown in FIG. 19A, the two photo-electric modules 111-1 and 111-2 are pasted as the photo-electric module 111-m on the distribution module 113. In this case, the module 111 is aligned on the module 113 to electrically connect an electrode for ground line 162 of the photo-electric module 111 and the electrode for ground line 167 of the distribution module 113 by solder. The electrode pad for data line 160 and the electrode pad for address line 161 are provided on the front surface of the photo-electric module 111. The electrode pad for ground line 162 is provided on the back surface thereof. Unlike Example 1, pasting is performed to the number of the photo-electric modules 111-m finally pasted onto the distribution module 113.

In the process 1-2 shown in FIG. 19B, wiring connection between the distribution module 113 and the last photo-electric module 111 is performed by wiring 144. Connection between the photo-electric modules 111 adjacent to each other is performed by the wiring 145. Wiring connection is performed for both the data line and the addressing line. In a position 192, the module wiring means 145 is provided between the photo-electric modules 111-1 and 111-2. In a position 193, the module wiring means 144 is provided between the last photo-electric module 111-2 and the distribution module 113.

In the process 1-3 shown in FIG. 19C, wiring protective layers (insulation resins) 122 for protecting the module wiring means 144 and 145 are formed. An integrated module 201 of the distribution module 113 and the photo-electric modules 111 is thus manufactured. The process 2-1 shown in FIG. 10 to the process 2-8 of FIG. 12B of Example 1 are performed to realize the X-ray detector 104 of this example.

### Example 3

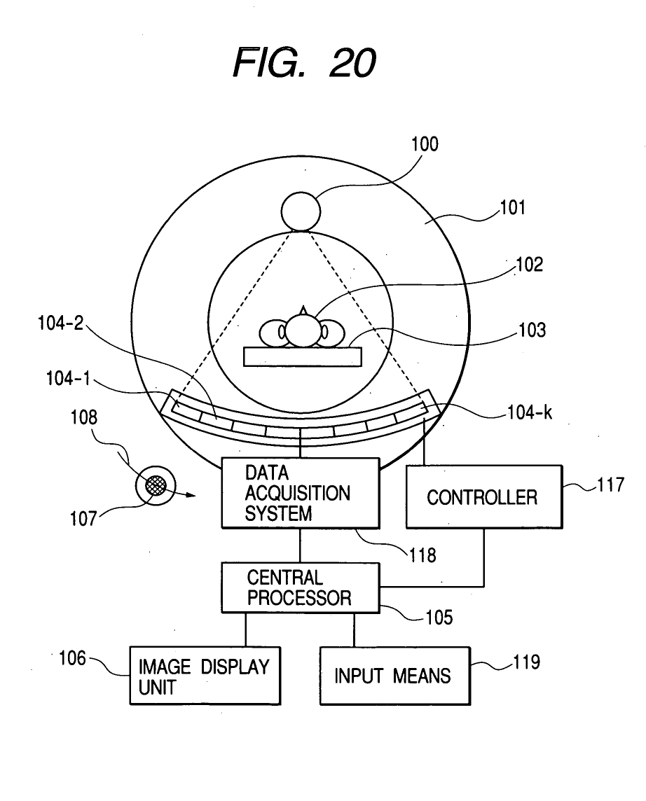

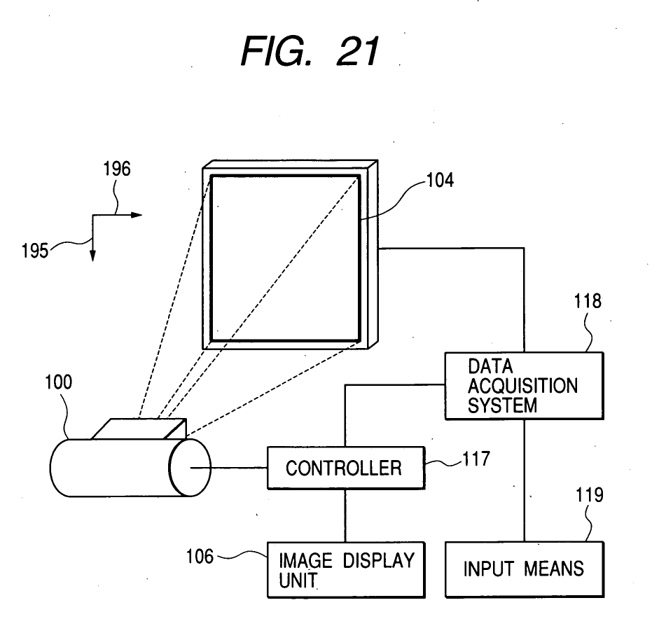

FIG. 20 shows an example of the configuration of the X-ray CT apparatus according to the present invention. The X-ray CT apparatus has an X-ray source 100, X-ray detectors 104, a data acquisition system (DAS) 118, a central processor 105, an image display unit 106, input means 119, a controller 117, a rotated gantry 101, and a bed 103.

The X-ray detectors 104 are described in Example 1 or 2. In FIG. 20, for simplifying the description, eight X-ray detectors are located in an arc. Actually, for example, 40 X-ray detectors are located.

One X-ray detector 104 is realized by pasting, in a slice direction, eight photo-electric modules 111 with X-ray detecting elements 110 in 24 columns in a slice direction 108 and in 256 lines in a slice direction 107. The size of the X-ray detecting element 110 is e.g., 1 mm×1 mm.

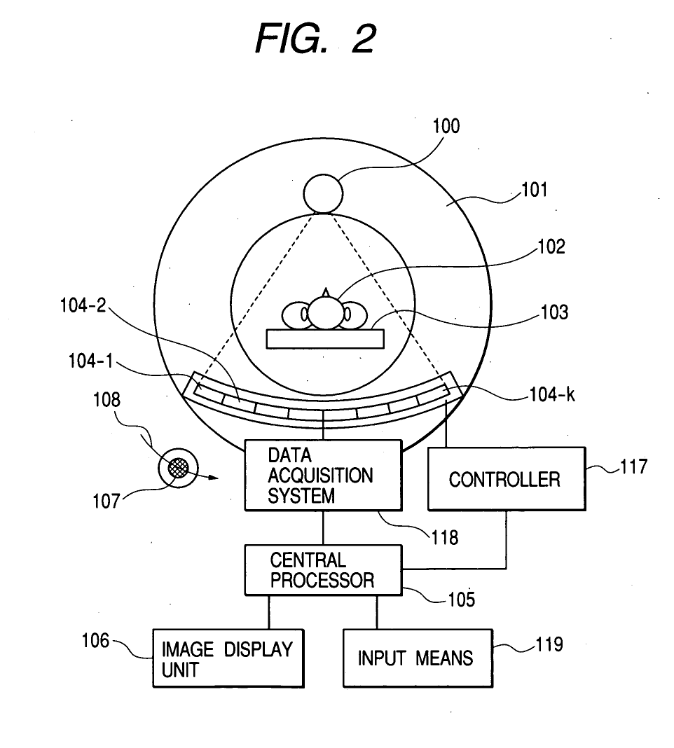

The X-ray source 100, the data acquisition system 118, the central processor 105, the image display unit 106, the input means 119, the controller 117, and the rotated gantry 103 have the same functions as those of the prior art X-ray CT apparatus described in FIG. 2 to obtain a tomographic image of an object 102.

The data acquisition system 118 or the central processor 105 has means correcting variation in sensitivity of the X-ray detecting elements 110. The correcting means is performed to a projection as analog data for each of the X-ray detecting elements 110-m-i-j by a circuit realizing operation as shown in the following equation (1).

(output value with correction)={(output value without correction)−(offset value)}/(sensitivity of detector) (1)

where the output value with correction is an output value after correcting the X-ray detecting element 110-m-i-j in a projection, the output value without correction is an output value before correcting the X-ray detecting element 110-m-i-j in the projection, the offset value is an output value of the X-ray detecting element 110-m-i-j when an X-ray is not irradiated, and the sensitivity of detector is a value in proportion to an electric signal produced when an X-ray is incident upon the X-ray detecting element 110-m-i-j.

The correcting means of this example is not limited to the method shown in the equation (1). The central processor 105 has the correcting means and may perform operation as shown in the following equation (2) to digital data after a projection is analog-to-digital converted (AD converted).

(output digital value with correction)={(output digital value without correction)−(offset level)}/(sensitivity level of detector) (2)

where the output digital value with correction is a digital output value after correcting the X-ray detecting element 110-m-i-j, the output digital value without correction is a digital value in which the output value of the X-ray detecting element 110-m-i-j, [the output value without correction in the equation (1)] is AD converted, the offset level is a value in which the output value of the X-ray detecting element 110-m-i-j is AD converted when an X-ray is not irradiated, and the sensitivity level of detector is a value in which an electric signal produced when an X-ray is incident upon the X-ray detecting element 110-m-i-j is AD converted.

A value necessary for correction is obtained to be separate from projection imaging. The data of the offset level is obtained by imaging plural projections without irradiating an X-ray to perform adding and averaging using the output values of the X-ray detecting elements 110-m-i-j after AD conversion.

The data of the sensitivity level of detector is obtained by irradiating a uniform X-ray onto the X-ray detectors 104 to image plural projections, performing adding and averaging using the output values of the X-ray detecting elements 110-m-i-j after AD conversion, and subtracting the offset level. The obtained data of the offset level and the sensitivity level of detector are stored into the central processor 105.

As another method for deciding a value necessary for correction, to calculate the offset level and the sensitivity level of detector, after calculating the offset level and the sensitivity level of detector of the X-ray detecting element 110-m-i-j, certain weighting and adding may be performed to them from the digital data values of the X-ray detecting elements 110 around it.

### Example 4

FIG. 20 shows an example of the configuration of the X-ray imaging system having the X-ray detectors 104 according to the present invention. The X-ray imaging system has an X-ray source 100, X-ray detectors 104, a data acquisition system (DAS) 118, an image display unit 106, input means 119, and a controller 117.

The X-ray detectors 104 have the construction described in Example 1 or 2. A plurality of photo-electric modules 111 are pasted in both directions of a vertical direction 195 and a horizontal direction 196. There is realized a flat panel detector having X-ray detecting elements 110 located in a two-dimensional (2D) manner in the vertical direction 195 and the horizontal direction 196.

The number of the X-ray detecting elements 110 has 512 columns in both the vertical direction 195 and the horizontal direction 196. The photo-electric modules 111 having the X-ray detecting elements 110 in 32 columns in both the vertical direction 195 and the horizontal direction 196 are pasted onto a circuit module 113 by 8 columns in both the vertical direction 195 and the horizontal direction 196.

The size of the X-ray detecting element 110 is e.g., 1 mm×1 mm. In imaging, an object is located between the X-ray source 100 and the X-ray detectors 104. When there is a start input from the input means 119, the controller 117 which has received the signal outputs an X-ray irradiation signal to the X-ray tube 100 and a start signal to the data acquisition system 118, irradiates an X-ray, and reads out a projection from the X-ray detectors 104 to the data acquisition system 118.

The projection thus obtained is AD converted by the data acquisition system 118. The data acquisition system 118 has correcting means and performs correcting processing correcting variation in sensitivity for each of the X-ray detecting elements 110 shown in the equation (2) for display on the image display unit 106.

### Modified Example

The present invention is not limited to the above examples and various modifications can be made and executed within the scope without departing from the purpose in the execution stage. The above examples include various stages and various inventions can be extracted by a suitable combination of plural components disclosed. Some of all the components shown in the examples may be removed.

The present invention can provide an X-ray detector which realizes a matrix construction having a large number of X-ray detecting elements by tiling and an X-ray CT apparatus and an X-ray imaging system onto which the same is mounted.

1. An X-ray detector comprising:
(1) an X-ray sensitive module having a plurality of X-ray detecting elements having a scintillator converting an X-ray to a light and transparent means optically connected to a light output surface of said scintillator transmitting an output light from said scintillator located integrally in a two-dimensional manner via optical reflecting means in a first and a second directions;
(2) a photo-electric module in which photo-electric means located in a two-dimensional manner corresponding to said transparent means of said X-ray detecting elements converting an output light outputted from said scintillator via said transparent means to an electric signal, a first data line reading out said electric signal, a first addressing line addressing said photo-electric means reading out said electric signal, and electrode pads forming part of said first data line or/and said first addressing line are formed, a light output surface of said transparent means is optically connected to said photo-electric means, the area of said photo-electric means positioned on the edge in said first direction is formed to be smaller than that of said photo-electric means positioned in other positions, said electrode pads are formed near an end surface on which said transparent means is not mounted, and a plurality of said X-ray sensitive modules are mounted to be adjacent to each other in said first or said second direction;
(3) a distribution module in which a second data line connected to said first data line reading out said electric signal and a second addressing line connected to said first addressing line addressing said photo-electric means reading out said electric signal are formed, and a plurality of said photo-electric modules are mounted; and
(4) module wiring means electrically connecting said electrode pads of said photo-electric modules adjacent to each other, or/and said electrode pad and said second data line, or/and said electrode pad and said second addressing line.

2. The X-ray detector according to claim 1, wherein said transparent means is made of a resin layer which has a thickness smaller than that of said scintillator, has optical transmittance higher than that of said scintillator and is stable to an X-ray, and has a shape in which an angle θ of a normal vector at an arbitrary point of a surface except for a light input surface from said scintillator and an output surface of said resin layer and a normal vector of said input surface or said output surface is 45°≦θ<90°.

3. The X-ray detector according to claim 2, wherein said resin layer is made of an epoxy resin layer.

4. An X-ray detector comprising:
(1) an X-ray sensitive module having a plurality of X-ray detecting elements having a scintillator converting an X-ray to a light and transparent means optically connected to an output surface of said scintillator transmitting an output light from said scintillator located integrally in a two-dimensional manner via optical reflecting means in a first and a second directions, said transparent means positioned on the edge in said first direction having a cutaway part in part thereof;
(2) a photo-electric module in which photo-electric means located in a two-dimensional manner corresponding to said transparent means of said X-ray detecting elements converting an output light outputted from said scintillator via said transparent means to an electric signal, a first data line reading out said electric signal, a first addressing line addressing said photo-electric means reading out said electric signal, and electrode pads forming part of said first data line or/and said first addressing line are formed, a light output surface of said transparent means is optically connected to said photo-electric means, the area of said photo-electric means positioned on the edge in said first direction is formed to be smaller than that of said photo-electric means positioned in other positions, said electrode pads are formed near an end surface on which said transparent means is not mounted, and a plurality of said X-ray sensitive modules are mounted to be adjacent to each other in said first or said second direction;
(3) a distribution module in which a second data line connected to said first data line reading out said electric signal and a second addressing line connected to said first addressing line addressing said photo-electric means reading out said electric signal are formed, and a plurality of said photo-electric modules are mounted; and
(4) module wiring means electrically connecting said electrode pads of said photo-electric modules adjacent to each other, or/and said electrode pad and said second data line, or/and said electrode pad and said second addressing line.

5. An X-ray CT apparatus comprising:
an X-ray tube generating an X-ray;
a plurality of X-ray detectors according to any one of claims 1 to 4 located in an arc in said second direction opposite said first X-ray tube;
a detector control circuit producing a control signal for addressing said photo-electric means reading out said electric signal of said X-ray detector and inputting it to said second addressing line;
a data acquisition system acquiring said electric signals outputted from said second data line to convert them to digital data;
arithmetic processing means performing arithmetic processing said digital data; and
an image display unit displaying the result of said arithmetic processing.

6. The X-ray CT apparatus according to claim 5, wherein said data acquisition system has data correcting means correcting said analog electric signal from said photo-electric means corresponding to part or all of said X-ray detecting elements, or said digital data obtained by converting said analog electric signal.

7. An X-ray imaging system comprising:
an X-ray tube generating an X-ray;
one or more X-ray detectors according to any one of claims 1 to 4 located opposite said X-ray tube;
a detector control circuit producing a control signal for addressing said photo-electric means reading out said electric signal of said X-ray detector and inputting it to a second addressing line;
a data acquisition system acquiring said electric signals outputted from said second data line to convert them to digital data; and
an image display unit displaying said digital data.

8. The X-ray imaging system according to claim 7, wherein said data acquisition system has data correcting means correcting said analog electric signal from said photo-electric means corresponding to part or all of said X-ray detecting elements of said X-ray detector, or said digital data obtained by converting said analog electric signal.
